(19) United States
(12) Patent Application Publication
(10) Pub. No.: US 2005/0059992 A1
(43) Pub. Date: Mar. 17, 2005

(54) Air introduction device for anastomotic leak testing

(76) Inventor: Arnold R. Leiboff, Stony Brook, NY (US)

Correspondence Address: STEINBERG & RASKIN, P.C., 1140 AVENUE OF THE AMERICAS, 15th FLOOR, NEW YORK, NY, 10036-5803, US

(21) Appl. No.: 10/664,366

(22) Filed: Sep. 17, 2003

(51) Int. Cl. A61M 29/00

(52) U.S. Cl. 606/197

(57) ABSTRACT

Air introduction device for anastomotic leak testing including an elastomeric body optimally configured for partial insertion into the rectum, occlusion of the anus and attachment to a commercially available insufflation bulb. The air introduction device may be dip molded in one piece to form a single use, disposable product. The body has a proximal portion insertable into an anus of a person to cause the anus to constrict around it and form a seal against the anal wall, a distal portion mating with the insufflation bulb to enable air to be directed therefrom into and through the body and an expanded portion which is larger than the proximal portion and interposed between the proximal portion and the distal portion. The expanded portion occludes the anus by engaging with the anal opening to limit insertion of the proximal portion into the anus and seal the body against the anal opening. Embodiments incorporating means to allow the insertion of an endoscope through the device for visual inspection of an anastomosis, and means to prevent over-inflation of the rectum are described.

### FIELD OF THE INVENTION

The present invention relates to a device for introducing air into the rectum of a patient during a surgical operation to remove the distal colon and part of the rectum in order to evaluate the integrity of an anastomosis, i.e., detect anastomotic leakage. The present invention also relates to methods for testing for anastomotic leakage and for visualization of a completed anastomosis.

### BACKGROUND OF THE INVENTION

Surgical operations to remove the distal colon and part of the rectum are common for illnesses such as neoplasia, diverticular disease and inflammatory bowel disease. When the continuity of the intestinal tract is to be preserved, the bowel must be reconnected by means of sutures, staples or a compression device. This connection is called an anastomosis.

If there is a defect in the anastomosis, bowel content can leak out of the bowel and contaminate the normally sterile peritoneal cavity, causing peritonitis. Peritonitis (infection of the peritoneal cavity) can be lethal, and therefore measures must be taken during surgery to ensure that leaks in the anastomosis are not present. One such measure is to fill the pelvic portion of the abdominal cavity with saline or water to a level which immerses the completed anastomosis, then insufflate the rectum with air while occluding the bowel above the anastomosis. As the rectum distends, the pool of saline is observed for air bubbles which, if present, signal a defect in the anastomosis, i.e., a leak, which must then be repaired.

One device currently used to insufflate air into the rectum is a proctoscope, i.e., a tubular instrument designed for looking into the rectum. The proctoscope has an insufflation bulb attached to it. With the lens gate of the proctoscope closed, air is introduced into the rectum by compressing the insufflation bulb.

Use of a proctoscope for rectal insufflation to test for anastomotic leaks has advantages and disadvantages. The fact that proctoscopes are items usually stocked in an operating room is an advantage. Thus, an additional surgical instrument does not need be purchased or stocked in an operating room in order to test for anastomotic leaks. Moreover, it is advantageous that it is possible to visually inspect the anastomosis from inside the bowel using the proctoscope. This helps identify potentially harmful bleeding, which can then be stopped by suture ligation.

On the other hand, a significant disadvantage of the use of a proctoscope results from the fact that a sterile proctoscope has to be contaminated to check for leaks and then repackaged and sterilized for future use. For example, if a proctoscope is used at the very start of surgery to examine a rectal tumor or cleanse the rectum, as is sometimes done, then it must either be kept in the operating room in a dirty state, or cleansed in a nearby utility room by a nurse, until such time as it is again needed to test the anastomosis. This not only clutters the operating room, but can potentially contaminate the operating room, and in any event, necessitates extra labor by a circulating nurse. The cost savings gained by using an instrument already in stock and accessible may be lost by the extra labor involved in maintaining the instrument in the operating room and repackaging and sterilizing the proctoscope after surgery.

An additional disadvantage of using a proctoscope to insufflate the rectum is that the insufflated air tends to leak out through the anus around the proctoscope, which typically has an outer diameter of about 0.75 inches. It therefore requires some extra effort to distend the rectum to the desired amount to effectively test the anastomosis. It is also possible to over-distend the rectum with air insufflation and actually disrupt the anastomosis. It is not possible to accurately gauge the level of air pressure in the rectum because the proctoscope does not provide any mechanism to measure the air pressure in the rectum, and thus the air pressure can only be grossly evaluated by visual inspection of the distended bowel above the pool of saline, or by feeling the bowel.

Yet another drawback of the use of a proctoscope is that although the proctoscope can be used to visually inspect the stapled anastomosis inside the bowel, the view obtained via a standard proctoscope is relatively poor, is not sufficiently keen to allow the surgeon to spot small anastomotic defects and usually requires the surgeon to crouch in an awkward position and spend some time to achieve a satisfactory inspection.

Furthermore, after performing an anastomosis and prior to testing for leaks, some surgeons prefer to cleanse the rectum with an antiseptic before they force air into the rectum and potentially into the sterile peritoneal cavity should there be an anastomotic leak. If a proctoscope which was used earlier, e.g., to cleanse the rectum, is reused without being resterilized, then they may insufflate the rectum with particles of fecal debris or airborne bacteria that remain within the scope or within the insufflation bulb or tubing which carries air from the insufflation bulb to the proctoscope. Such fecal debris or airborne bacteria could contribute to the development of postoperative peritonitis or wound infection.

Thus, there are significant drawbacks to the use of a standard proctoscope to insufflate air into the rectum and to inspect an anastomosis and it would be desirable to provide alternative means to insufflate the rectum and inspect the anastomosis which avoid these drawbacks.

### OBJECTS AND SUMMARY OF THE INVENTION

It is an object of the present invention to provide a simple, inexpensive and disposable device to be used during surgery for insufflation of the rectum with air to test for anastomotic leakage.

It is another object of the present invention to provide new methods for testing for anastomotic leaks using a novel air introduction device.

It is still another object of the present invention to provide an anastomotic leak tester including a novel air introduction device which is easier to use than a proctoscope for the purpose of testing an anastomosis for leaks.

It is yet another object of the present invention to provide an air introduction device for testing for anastomotic leaks which is safer to use than a standard proctoscope.

Another object of the present invention is to provide an inexpensive anastomotic leak tester that can lead to cost savings and more efficient use of operating room time and personnel.

Another object of the present invention is to provide a new anastomotic leak tester which provides a signal when there is sufficient air pressure in the rectum.

Still another object of the present invention is to provide a new and improved anastomotic leak tester which enables improved visualization of the anastomosis from within the bowel.

In order to achieve these objects and others, an air introduction device for use in anastomotic leak testing in accordance with the invention comprises an elastomeric tube, optimally configured for partial insertion into the rectum, occlusion of the anus and attachment to a commercially available pumping device such as an insufflation bulb. The air introduction device may be dip molded in one piece so that it would be simple and inexpensive to manufacture and ideal for a single use, disposable product.

More specifically, the air introduction device for use in anastomotic leak testing comprises a unitary, elastomeric body defining an interior space and having a proximal portion adapted to be inserted into an anus of a person to cause the anus to constrict around it and thereby form a seal against an anal wall, a distal portion adapted to mate with a pumping device, such as an insufflation bulb, to enable air to be directed from the pumping device into and through the body and an expanded portion having a larger size than the proximal portion and interposed between the proximal portion and the distal portion. The expanded portion occludes the anus by being adapted to engage with an anal opening to limit insertion of the proximal portion into the anus and seal the elastomeric body against the anal opening.

In one embodiment, the distal portion includes only a single arm, i.e., a single tubular segment defining a lumen adapted to receive the insufflation bulb. In this case, the distal portion has an outer diameter smaller than the outer diameter of the proximal portion.

In another embodiment, the distal portion has two arms, one arm defining a lumen adapted to receive a connector of the insufflation bulb and the other arm defining a lumen through which a visualization device or endoscope such as a laparoscope may be inserted when the distal end is changed from a closed form to an open form. Thus, one arm serves as an insufflation arm and the other serves as an endoscopic port.

Instead of or in addition to an arm providing an endoscopic port, an arm can be provided to retain a pressure relief valve for releasing air when a specific air pressure in the rectum is reached. This prevents over-inflation of the rectum. A signal mechanism can be associated with the valve to provide a signal when air is released via the valve, e.g., an extension coupled to the arm and including vibrating flaps which vibrate and produce an audible sound when air is released via the valve. Alternatively, the functions of a pressure relief valve and signal mechanism can be incorporated into a single element, whose flaps will separate and vibrate only when a critical air pressure within the rectum is reached.

Another way to consider the air introduction device in accordance with the invention would be as a unitary body defining an interior space and having an insertion and sealing mechanism for enabling insertion of a part of the body into an anus of a person such that the anus constricts around the part and thereby seals the body against anal walls, an insertion-limiting mechanism for limiting insertion of the part of the body into the anus and occluding the opening of the anus, and a coupling mechanism for enabling coupling of the body to an insufflation bulb such that air is directable from the insufflation bulb through the coupling mechanism into the interior space in the elastomeric body. The insertion and sealing mechanism may be the proximal portion of the body discussed above. The insertion-limiting and anal occluding mechanism may be the expanded portion of the body discussed above. The coupling mechanism may be a lumen arranged on a distal portion of the body and adapted to receive a connector of the insufflation bulb.

The distal portion can include, in addition to a lumen in an arm for receiving a connector of an insufflation bulb, an arm defining a lumen through which a visualization device or endoscope such as a laparoscope may be inserted and/or an arm having a pressure relief mechanism for releasing air when a specific air pressure in the rectum is reached, with or without a signal mechanism.

An exemplifying method for anastomotic leak testing in accordance with the invention includes the steps of providing a unitary body defining an interior space and having a proximal portion, a distal portion and an expanded portion interposed between the proximal portion and the distal portion, coupling an insufflation bulb to the distal portion of the body, and inserting the proximal portion of the elastomeric body into the anus of a patient to cause the proximal portion to dilate the anus whereby the anus constricts around the proximal portion and a seal is formed between the proximal portion and the wall of the anus to prevent escape of air from the rectum, and the expanded portion presses against the opening of the anus also to prevent the escape of air from the rectum. The pelvis of the patient is filled with fluid, the insufflation bulb is repeatedly compressed to distend the rectum of the patient with air and a check for anastomotic leaks is performed based on the presence of air bubbles in the pelvic fluid once the rectum is sufficiently distended with air. The insufflation bulb may be detached from the body after checking for anastomotic leaks while the proximal portion of the body remains inserted in the anus thereby allowing air from the rectum to escape from the rectum and the rectum to deflate. The body is then removed from the anus.

The body may include a second arm having a lumen through which a visualization device or endoscope such as a laparoscope may be inserted. Initially, the end of the second arm is closed but after the initial anastomotic leak check described above is performed, the body is removed from the anus and the closed end of the second arm is opened. An endoscope may then be inserted into the lumen so that the tip of the endoscope sits within a space of the elastomeric body. The proximal portion of the body is re-inserted into the anus and the insufflation bulb is again compressed to distend the rectum of the patient with air. The anastomosis is then visually inspected by advancing the tip of the endoscope from the space in the elastomeric body into the rectum until the anastomosis is viewable through the endoscope or on a video display to thereby enable the integrity of the anastomosis to be ascertained and to check for bleeding at the anastomosis. The endoscope is removed from the elastomeric body when the visual inspection is complete while the body is engaged with the anus and then the rectum is deflated and the body removed out of engagement with the anus.

An air introduction device for anastomotic leak testing in accordance with the invention, and method for using the same, has several advantages over the prior art, notably the use of a standard proctoscope for the purpose of insufflating the rectum to test for anastomotic leakage. An anastomotic leak tester including the air introduction device is easier to use than a proctoscope because it is more compact and requires only one hand to operate, whereas a proctoscope is relatively unwieldy and requires two hands to use, one to position the proctoscope and the second to repetitively compress the insufflation bulb. The device in accordance with the invention is also easier to use than a proctoscope because it prevents air from escaping from the rectum around the device, resulting in fewer insufflation bulb compressions to achieve the same rectal distension.

An anastomotic leak tester including the air introduction device in accordance with the invention is also safer to use than a standard proctoscope because the device is sterile until used to insufflate the rectum and therefore no potentially hazardous debris collects in the device nor is there a possibility of debris originating from the device contaminating the peritoneal cavity (as is the case when a contaminated proctoscope is used).

The anastomotic leak tester is also designed to be an inexpensive alternative to a proctoscope and to be easier to open, use and dispose of than it would be to cleanse a proctoscope, transport it, repackage it and sterilize it again.

One embodiment of the anastomotic leak tester also prevents the surgeon from inflating the rectum above a desired pressure by providing a pressure relief valve.

Another embodiment provides a signal when a desired air pressure within the rectum is reached.

Another embodiment of the anastomotic leak tester is designed for use in conjunction with a laparoscope or other image-obtaining device that can visualize the anastomosis from within the bowel to a degree of clarity which far surpasses the view that one can achieve with a rigid proctoscope.

### DESCRIPTION OF THE DRAWINGS

The invention, together with further objects and advantages thereof, may best be understood by reference to the following description taken in conjunction with the accompanying drawings wherein like reference numerals identify like elements.

FIG. 1 is a perspective view of a first embodiment of an air introduction device for use in anastomotic leak testing in accordance with the invention.

FIG. 2 is a cross-sectional view of the embodiment shown in FIG. 1.

FIG. 3 is a perspective view of an insufflation bulb for use in combination with the air introduction device in accordance with the invention.

FIG. 4 is a perspective view of the first embodiment of the air introduction device in accordance with the invention shown coupled to the insufflation bulb shown in FIG. 3 to form an anastomotic leak tester in accordance with the invention.

FIG. 5 is a diagram showing the use of the anastomotic leak tester of FIG. 4 inserted into the anus of a patient for testing of an anastomosis.

FIG. 6 is a perspective view of a second embodiment of an air introduction device for use in anastomotic leak testing in accordance with the invention.

FIG. 7 is a cross-sectional view of the embodiment shown in FIG. 6.

FIG. 8 is a perspective view of the second embodiment of the air introduction device in accordance with the invention shown coupled to the insufflation bulb shown in FIG. 3 to form an anastomotic leak tester in accordance with the invention.

FIG. 9 is a perspective view of the anastomotic leak tester of FIG. 8 showing a laparoscope inserted through the endoscopic port.

FIG. 10 is a diagram showing the use of the anastomotic leak tester of FIG. 9 inserted into the anus of a patient for inspection of an anastomosis.

FIG. 11 is a cross-sectional drawing of a third embodiment of an air introduction device in accordance with the invention.

FIG. 12 is a cross-sectional drawing of a fourth embodiment of an air introduction device in accordance with the invention.

### DETAILED DESCRIPTION OF THE INVENTION

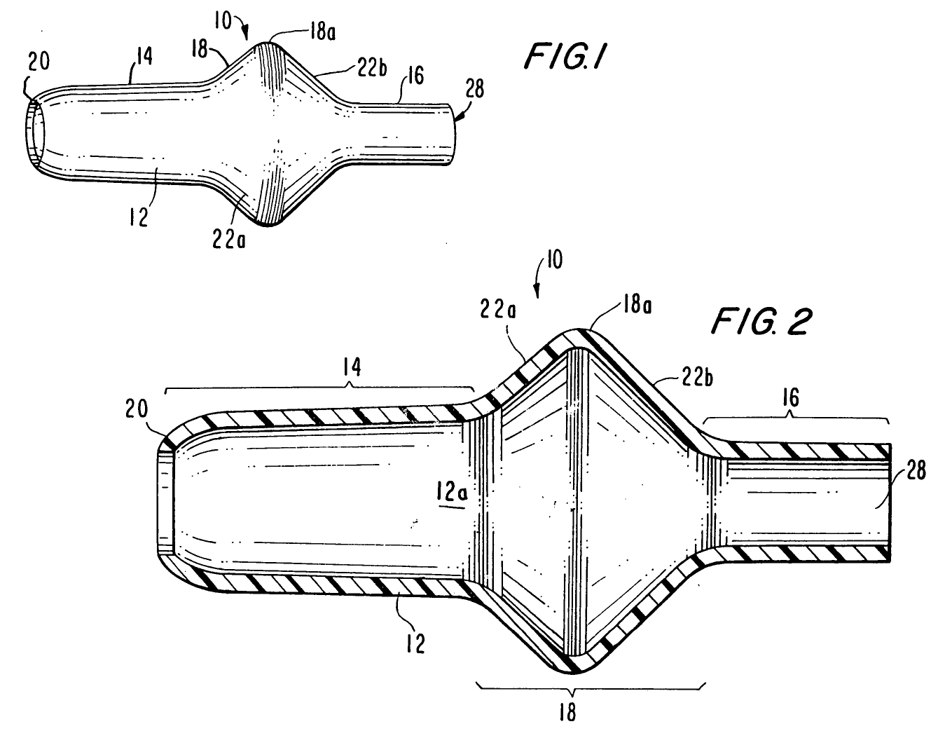

Referring to the accompanying drawings wherein like reference numerals refer to the same or similar elements, FIGS. 1 and 2 show a first embodiment of an air introduction device for anastomotic leak testing in accordance with the invention which is designated generally as 10. The air introduction device 10 comprises a substantially tubular, unitary body 12 defining an interior space 12a and has a proximal segment or portion 14 and a distal segment or portion 16 separated from the proximal portion 14 by an expanded segment or portion 18.

The body 12 of the air introduction device 10 may be made of an elastomer compatible for contact with internal parts of the human body, namely the anal wall and anal opening as discussed below, and fabricated using a dip molding technique to obtain a soft, pliable form. Other plastic manufacturing methods can also be used.

Proximal portion 14 has a substantially uniform inner and outer diameter with the exception that a tip 20 of the proximal portion 14 is rounded or tapered to aid insertion of proximal portion 14 into the anus of an anesthetized patient. The distal portion 16 has a substantially uniform inner and outer diameter smaller than those of the proximal portion 14. Expanded portion 18 has an outer circumferential portion 18a having the largest diameter of the entire body 12 and which is dimensioned to be larger than the opening of most human anuses when they are maximally dilated so that the body 12 can't be inserted into the anus farther than the outer circumferential portion 18a. That is, the expanded portion 18 serves as insertion-limiting flange which serves to limit the amount of insertion of the proximal portion 14 of the body 12 into the anus. The expanded portion 18 includes two truncated conical surfaces 22a, 22b, one surface 22a tapering from the outer circumferential portion 18a toward the rear edge of the proximal portion 14 and one surface 22b tapering from the outer circumferential portion 18a toward the front edge of the distal portion 16.

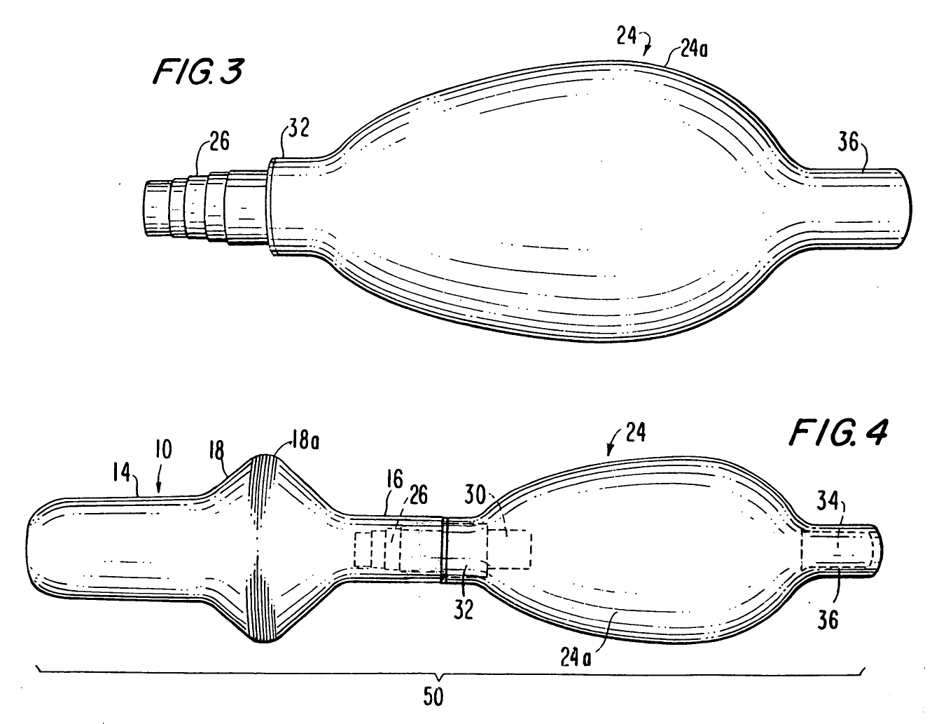

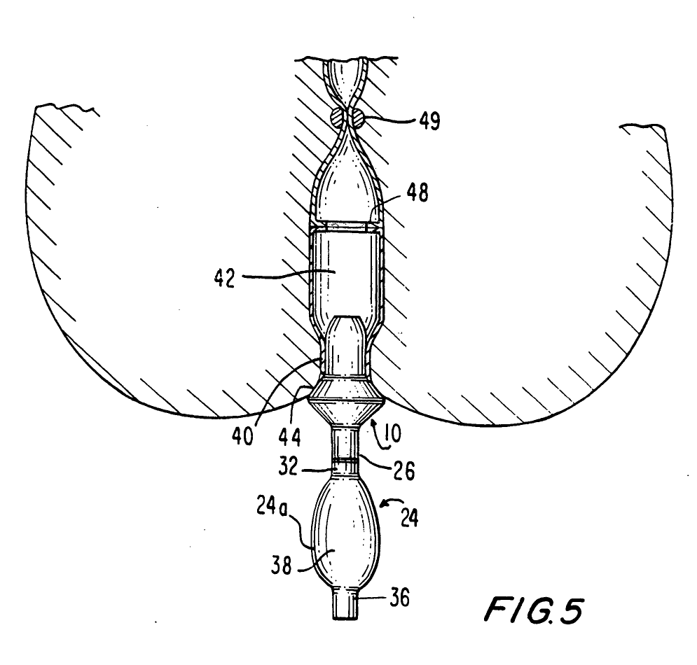

The air introduction device 10 is designed for use with a pumping device such as an insufflation bulb 24 shown in FIG. 3. The insufflation bulb 24 has a central tubular portion 24a defining an interior space receivable of air and is coupled to the distal portion 16 of the body 12 (see FIG. 4). More specifically, the insufflation bulb 24 includes a connector 26 which is insertable into a lumen 28 defined by the distal portion 16 of the air introduction device 10. The insufflation bulb 24 also includes a valve 30 at a proximal end 32 (housed within the connector 26 of the insufflation bulb 24 as shown in FIG. 5) and a valve 34 at a distal end 36 (housed within the insufflation bulb 24). The valves 30,34 permit movement of air in one direction only, specifically in a direction from the distal end 36 to the proximal end 32.

A central portion 24a of the insufflation bulb 24 between the valves 30,34 is compressible and when compressed, forces air from a space 38 defined in the interior of the insufflation bulb 24 through the valve 30 at the proximal end 32 and through the connector 26 (and into the air introduction device 10 when coupled to the insufflation bulb 24). When the compressing force on the central portion 24a of the insufflation bulb 24 is released, air flows into the space 38 through the valve 34 at the distal end 36. Upon the next compression of the central portion 24a of the insufflation bulb 24, this air is then directed through the connector 26 into the air introduction device 10. As such, a pumping action is obtained upon repeated compressions and releases of the central portion 24a of the insufflation bulb 24.

Referring now to FIG. 5, in one exemplifying use to test whether an anastomosis 48 is leak-proof, the insufflation bulb 24 is coupled to the air introduction device 10 by inserting connector 26 into the lumen 28 of the distal portion 16 of the air introduction device 10 to thereby form the anastomotic leak tester 50 shown in FIG. 4. The proximal portion 14 of the air introduction device 10 is then inserted into the anus 40 of an anesthetized patient (see FIG. 5). The pelvis is filled with fluid and the bowel above the anastomosis is occluded with a clamp 49. Repeatedly compressing the insufflation bulb 24 distends the rectum 42 with air. Air cannot escape from the anus 40 because the proximal portion 14 is dimensioned such that its walls dilate the anus 40 whereby the elastic anus constricts around the proximal portion 14 of the air introduction device 10 to form a seal between the walls of the proximal portion 14 and the walls of the anus 40. Thus, air cannot pass between the outer wall of the air introduction device 10 and the wall of the anus 40.

Furthermore, as the rectum 42 is being insufflated by the repeated compression of the insufflation bulb 24, the air introduction device 10 is pushed inward until the conical surface 22a of the expanded portion 18 presses against the anal verge or opening 44, adding a further seal to prevent the escape of air from the rectum 42. When the rectum 42 is sufficiently distended with air, and no air bubbles are seen in the pelvic fluid, it can be considered that the anastomosis 48 is air tight.

The insufflation bulb 24 is then detached from the air introduction device 10 while the proximal portion 14 thereof remains in the anus 40, which allows air from the rectum 42 to escape, and allows the rectum 42 to deflate. The air introduction device 10 is then removed out of engagement with the anus 40.

In one exemplifying embodiment, the outer diameter of the walls defining the proximal portion 14 is approximately 1.062 inches, which has been determined to be suitable to allow it to be inserted into the anal canal of an anesthetized patient with ease and at the same time prevent the escape of air from the rectum 42 by passing between the wall of the air introduction device 10 and wall of the anus 40. The largest diameter of the expanded portion 18, i.e., the outer circumferential portion 18a, has an approximate outer diameter of 1.75 inches which is too large to enter the anal canal (without applying excessive force) and which thus abuts and occludes the anal opening 44 when the proximal portion 14 is inserted completely into the anus 40. The expanded portion 18 thus serves to both limit the extent to which the air introduction device 10 is inserted into the rectum 42 and occlude the anal opening 44 to prevent leakage of air around the air introduction device 10. The distal portion 16 has an inner diameter of approximately 0.375 inches, which joins easily with an inexpensive disposable insufflation bulb 24, which is commercially available, by means of the tubing connector 26 of the insufflation bulb 24.

These dimensions, while allowing for optimal performance, also permit the air introduction device to be formed by a dip molding process, which is less expensive than other molding processes (e.g. injection molding). If the proximal portion 14 were appreciably thinner, or if the maximum diameter 18a of the expanded portion 18 were appreciably larger, the mandrel, around which the elastomeric device is molded, could not be readily removed from the device (unless the walls of the device were made thinner, which would render the device too flimsy for use.)

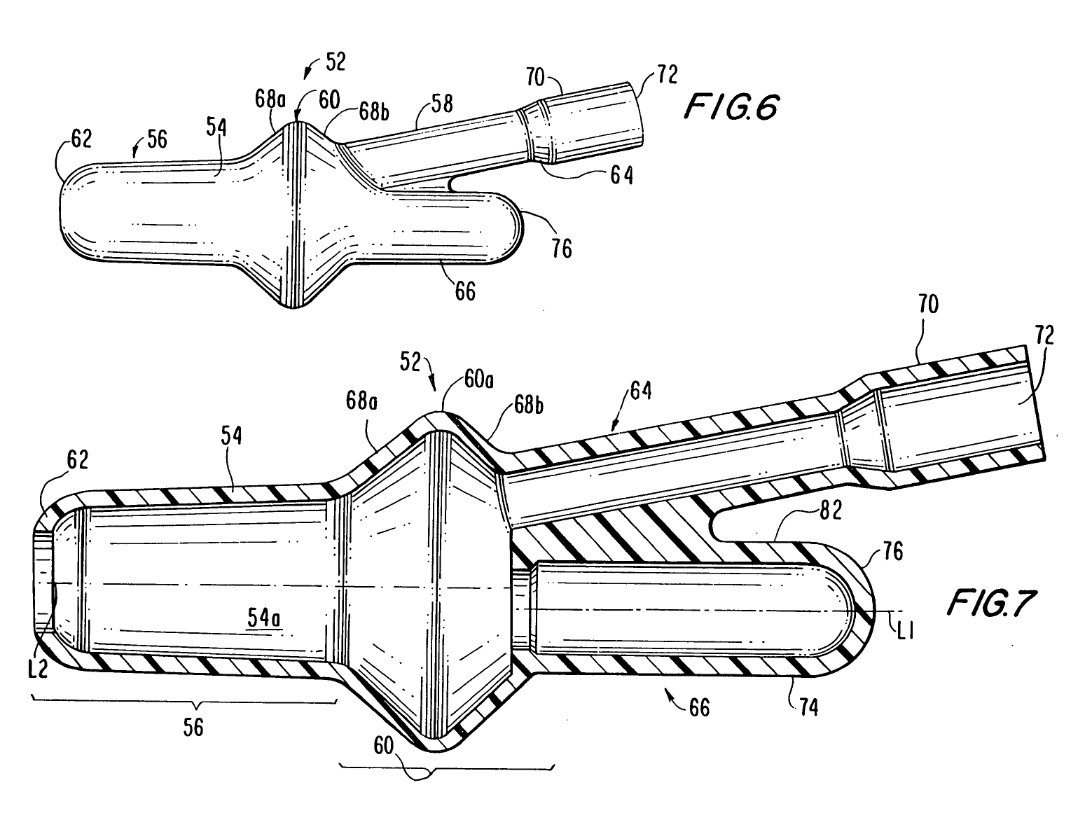

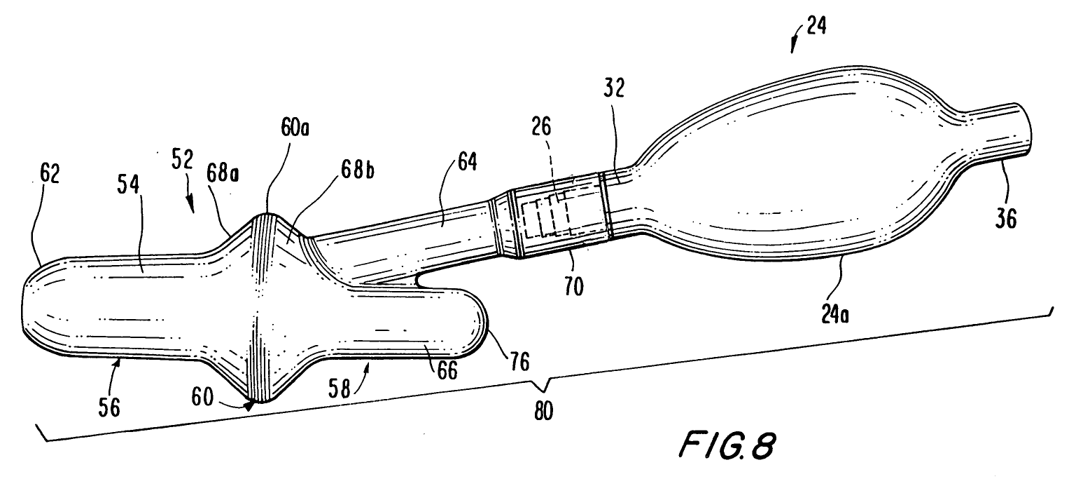

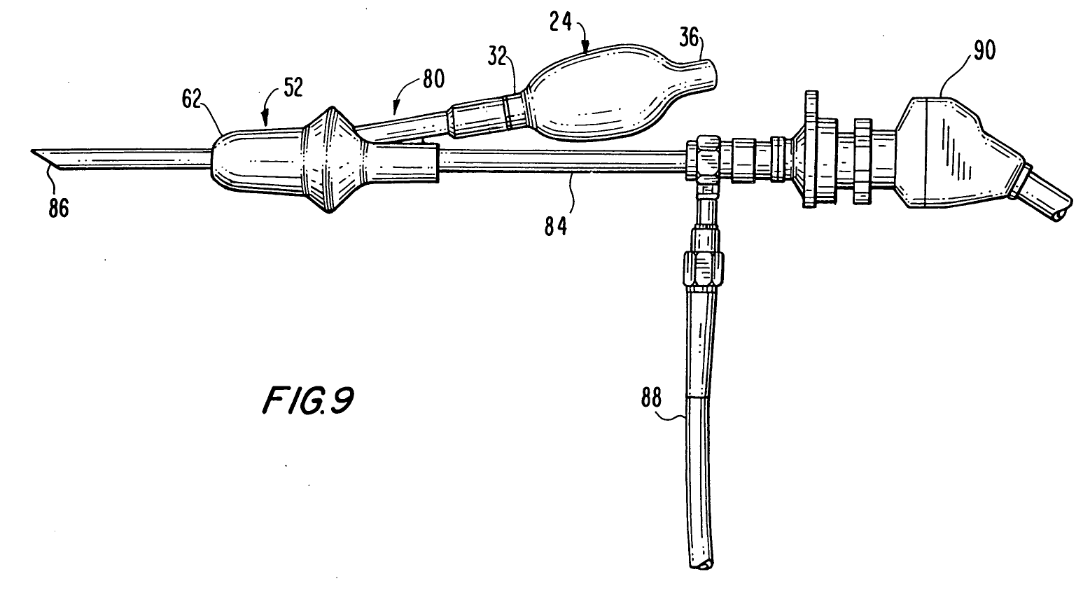

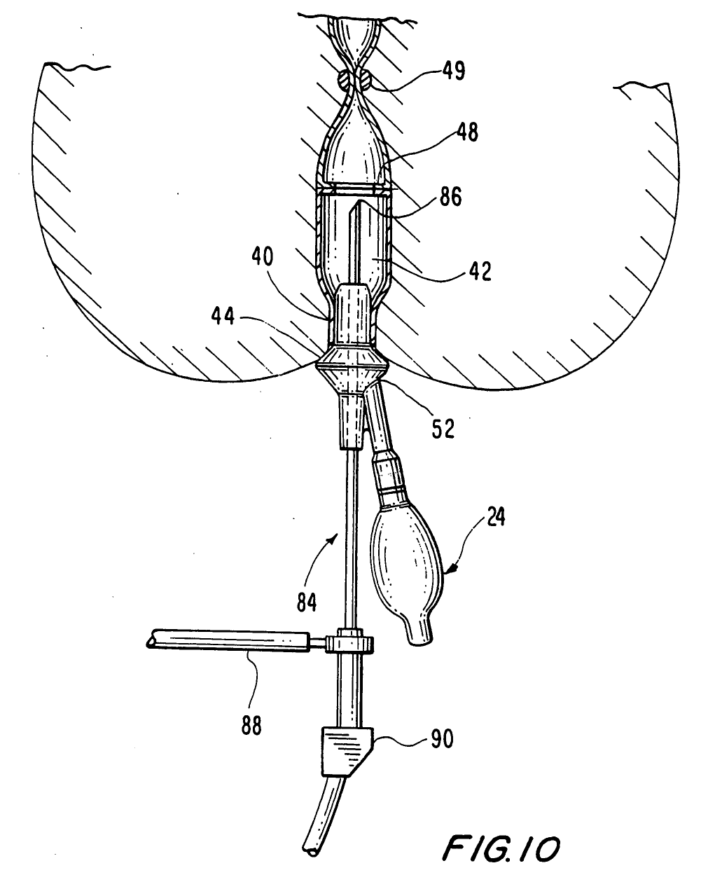

Referring now to FIGS. 6-10, a second embodiment of an air introduction device in accordance with the invention is designated generally as 52 and comprises a unitary body 54 defining an interior space 54a and having a proximal segment or portion 56 and a distal segment or portion 58 separated from the proximal portion 56 by an expanded segment or portion 60. The body 54 of the air introduction device 52 may be made of an elastomer compatible for contact with internal parts of the human body and fabricated using a dip molding technique to obtain a soft, pliable form. Other plastic manufacturing methods can also be used.

The proximal portion 56 has a substantially uniform inner and outer diameter with the exception that a tip 62 is rounded or tapered to aid insertion of proximal portion 56 into the anus of an anesthetized patient. The distal portion 58 has two arms 64,66 extending from the expanded portion 60. Expanded portion 60 has an outer circumferential portion 60a having the largest diameter thereof and which is dimensioned to be larger than the opening of most human anuses when they are maximally dilated so that the air introduction device 52 cannot be inserted into the anus farther than the circumferential portion 60a. The expanded portion 60 includes two truncated conical surfaces 68a, 68b, one surface 68a tapering from the outer circumferential portion 60a toward the rear edge of the proximal portion 56 and one surface 68b tapering from the outer circumferential portion 60a toward the front edge of the distal portion 58.

The first arm 64 on the distal portion 58 constitutes an insulation side arm defining an insufflation port whereas the second arm 66 constitutes an endoscopic side arm defining an endoscopic port. More specifically, the first arm 64 has a distal end 70 and lumen 72 which is designed to receive the connector 26 of the insufflation bulb 24 (the same as shown in FIG. 3) and communicates with the interior space 54a of the body 54. The second arm 66 defines a lumen 74 between the interior space 54a and a closed end 76. The lumen 74 is separated from the interior space 54a by a constriction 78, which may be approximately 10 mm in diameter. In the non-limiting illustrated embodiment, a longitudinal axis of the second arm 66 (designated L1) is parallel to a longitudinal axis of the proximal portion 56 and the expanded portion 60 (designated L2), but offset therefrom.

The air introduction device 52 may include the same features as the air introduction device 10 described above, to the extent possible.

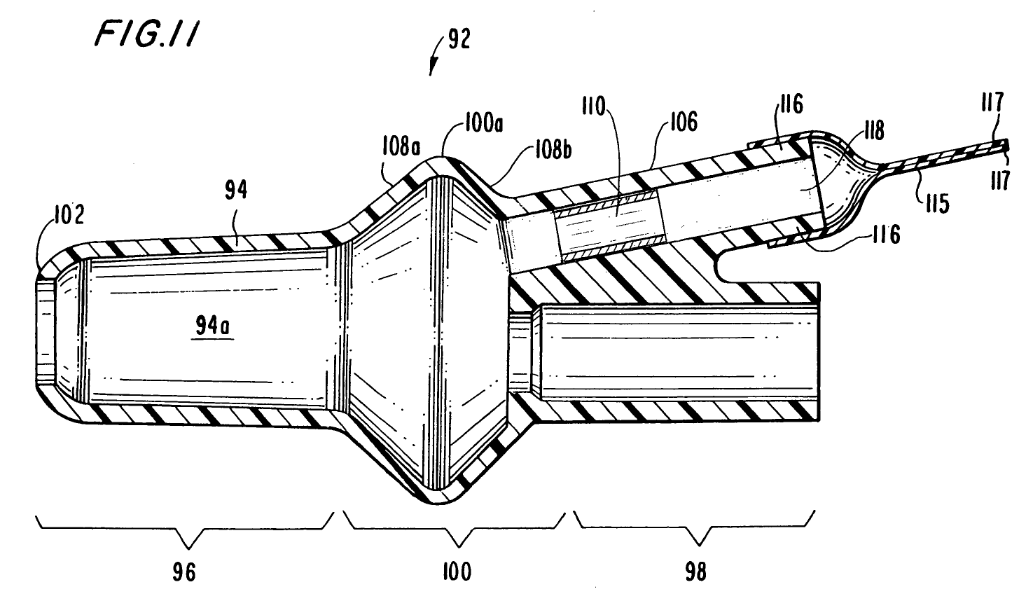

In one exemplifying use to test whether an anastomosis 48 is leak-proof, the air introduction device 52 is coupled to the insufflation bulb 24 by inserting connector 26 of the insufflation bulb 24 into lumen 72 of the distal portion 58 of the air introduction device 52 to thereby form the anastomotic leak tester 80 shown in FIG. 8. The proximal portion 56 of the air introduction device 52 is then inserted into the anus 40 of an anesthetized patient (see FIG. 10). The pelvis is filled with fluid and the bowel above the anastomosis is occluded with clamp 49. The insufflation bulb 24 is compressed several times, distending the rectum 42 with air. As explained above, air cannot escape from the anus 40 because of the seals formed by the proximal portion 56 of the air introduction device 52 against the anal wall and the expanded portion 60 of the air introduction device 52 against the anal opening 44. When the rectum 42 is sufficiently distended with air, and no air bubbles are seen in the pelvic fluid, it is can be considered that the anastomosis 48 is air tight and leak-proof. The air introduction device 52 is then removed from the anus.

A second way to evaluate the anastomosis involves a visual inspection of the internal aspect of the anastomosis 48. To this end, the closed end 76 of the second arm 66 is severed off, for example, with a scissors at point 82 between the closed end 76 and a location where the second arm 66 joins the first arm 64.

A laparoscope 84 is then inserted into lumen 74 so that the tip or proximal end 86 of the laparoscope 84 sits within space 54a of the body 54. The laparoscope 84 is attached to a fiberoptic cable 88 which is connected to a light source (not shown). Laparoscope 84 may be attached to a camera 90 which is connected to an image processor (not shown) and video display (not shown). The proximal portion 56 of the air introduction device 52 (attached to the insufflation bulb 24) is then inserted into the anus 40 so that the expanded portion 60 abuts against the anal verge 44. The insufflation bulb 24 is compressed several times, distending the rectum 42 with air. The tip 86 of the laparoscope 84 is then advanced from the position in the space 54a in the elastomeric body 54 into the rectum 42 while viewing the advancing progress directly through the lense of the laparoscope or on a video display to a point that permits a clear picture of the anastomosis 48 (see FIG. 10).

The constriction 78 serves to provide a seal against the laparoscope 84 to prevent air and fluid in the interior space 54a from being released from the body 54 via the lumen 74.

A laparoscope with an angled tip may be used, and the laparoscope may be rotated to obtain an excellent view of the inner aspect of the bowel anastomosis 48 on the video display. Bleeding from the anastomosis 48 can be easily identified and anastomotic defects visualized. Corrective surgical action can then be taken.

When the visual inspection is complete, the laparoscope 84 is removed with the leak tester 80 remaining in place. The rectum 42 is deflated and then the leak tester 80 removed out of engagement with the patient.

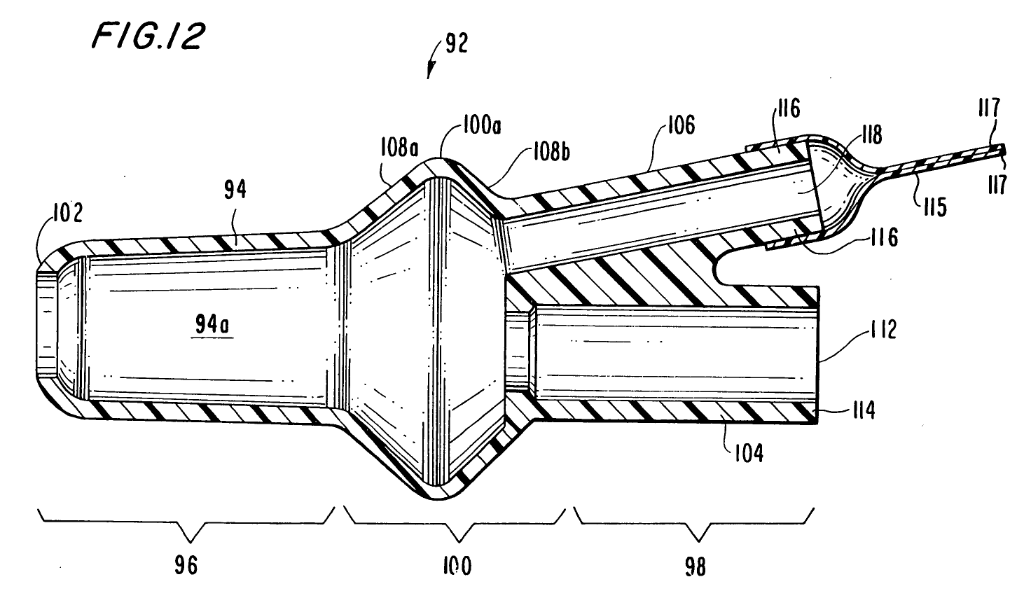

Referring now to FIG. 11, a third embodiment of an air introduction device in accordance with the invention is designated generally as 92 and comprises a unitary body 94 defining an interior space 94a and having a proximal segment or portion 96 and a distal segment or portion 98 separated from the proximal portion 96 by an expanded segment or portion 100. The body 94 of the air introduction device 92 may be made of an elastomer compatible for contact with internal parts of the human body and fabricated using a dip molding technique to obtain a soft, pliable form. Other plastic manufacturing methods can also be used.

The proximal portion 96 has a substantially uniform inner and outer diameter with the exception that a tip 102 is rounded or tapered to aid insertion of proximal portion 96 into the anus of an anesthetized patient. The distal portion 98 has two arms 104, 106 extending from the expanded portion 100. Expanded portion 100 has an outer circumferential portion 100a having the largest diameter thereof and which is dimensioned to be larger than the opening of most human anuses when they are maximally dilated so that the air introduction device 92 cannot be inserted into the anus farther than the circumferential portion 100a. The expanded portion 100 includes two truncated conical surfaces 108a, 108b, one surface 108a tapering from the outer circumferential portion 100a toward the rear edge of the proximal portion 96 and one surface 108b tapering from the outer circumferential portion 100a toward the front edge of the distal portion 98.

The first arm 104 on the distal portion 98 constitutes an insufflation side arm defining an insufflation port whereas the second arm 106 includes a pressure relief valve 110 arranged in a lumen 118. The first arm 104 has a distal end 112 and a lumen 114 which is designed to receive the connector 26 of the insufflation bulb 24 (the same as shown in FIG. 3) and communicates with the interior space 94a of the body 94.

Valve 110 is a pressure relief valve which allows air to escape from the rectum 42 when a specific air pressure within the rectum 42 is reached. This prevents over-inflation of and damage to the rectum 42. A bill-shaped extension 115 is attached to a distal end 116 of the second arm 106 and may be separately formed from an elastic material such as rubber and fixed to the distal end 116 of the second arm 106 by an adhesive or other comparable attachment mechanism.

When air escapes the rectum 42 through the second arm 106, i.e., through valve 110, it passes through extension 115 causing upper and/or lower flaps 117 of the extension 115 to vibrate. This produces an audible signal which alerts the surgeon to the fact that the desired air pressure within the rectum 42 has been reached. Other mechanisms for causing an audible (e.g. a whistle) or visual indication when air is released via pressure relief valve 110 can be used instead of the flaps 117.

Alternatively, the extension 115 may be designed to permit passage of air between the flaps 117 only when a specific air pressure is reached in the rectum. That is, the extension 115 may function both as a pressure release valve and a mechanism to produce an audible signal. In this case, a separate pressure relief valve 110 is not necessary and not provided, as shown in FIG. 12.

The air introduction device 92 may include the same features as the air introduction devices 10, 52 described above, to the extent possible.

In another embodiment of the invention, the distal portion of the body may include three arms, an insufflation port on one arm (for mating with an insufflation bulb or other pumping device), an endoscopic port on another arm (for enabling passage of an endoscope therethrough) and a third arm containing a pressure relief valve and signal producing extension.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

1. An air introduction device for use in anastomotic leak testing, comprising:
a unitary, elastomeric body defining an interior space and having a proximal portion adapted to be inserted into an anus of a person such that said proximal portion causes the anus to constrict around said proximal portion and thereby seal said proximal portion against the anal wall, a distal portion adapted to mate with a pumping device to enable air to be directed from the pumping device into and through said body and an expanded portion having a larger size than said proximal portion and interposed between said proximal portion and said distal portion, said expanded portion being adapted to engage with an anal opening to limit insertion of said proximal portion into the anus and seal said body against the anal opening.

2. The air introduction device of claim 1, wherein said proximal portion includes a rounded or tapered tip to facilitate insertion of said proximal portion into the anus.

3. The air introduction device of claim 1, wherein said expanded portion includes a first truncated conical surface tapering from a circumferential portion having a largest diameter of said expanded portion to said proximal portion and a second truncated conical surface tapering from said largest diameter circumferential portion of said expanded portion to said distal portion.

4. The air introduction device of claim 1, wherein said distal portion defines a lumen adapted to receive a connector of an insufflation bulb.

5. The air introduction device of claim 1, wherein said body is substantially tubular, said proximal portion being defined by a wall having an outer diameter of about 1.062 inches, said expanded portion being defined by a wall having a maximum outer diameter of about 1.75 inches and said distal portion being defined by a wall having an inner diameter of about 0.375 inches.

6. The air introduction device of claim 1, wherein said distal portion has first and second arms, said first arm defining a first lumen adapted to receive a connector of an insufflation bulb, said second arm defining a second lumen and having a closed distal end, said second lumen being adapted to receive a visualization device when said distal end is opened.

7. The air introduction device of claim 6, wherein said second arm includes a constriction between said second lumen and said interior space of said body.

8. The air introduction device of claim 6, wherein said proximal portion and said expanded portion have a common central axis and said second arm of said distal portion has a central axis parallel to and offset from said common central axis of said proximal portion and said expanded portion.

9. The air introduction device of claim 1, wherein said distal portion has first and second arms, said first arm defining a first lumen adapted to receive a connector of an insufflation bulb, said second arm defining a second lumen, further comprising a pressure relief valve arranged in said second lumen for releasing air when a specific air pressure in the rectum is reached.

10. The air introduction device of claim 9, further comprising signal means coupled to said second side arm for providing a signal when air is released via said valve.

11. The air introduction device of claim 10, wherein said signal means comprise a bill-shaped extension attached to said second arm and having at least one flap arranged to vibrate and produce an audible signal when air is released via said valve.

12. The air introduction device of claim 1, wherein said distal portion has first and second arms, said first arm defining a first lumen adapted to receive a connector of an insufflation bulb, said second arm defining a second lumen, further comprising a bill-shaped extension attached to said second arm and having at least one flap arranged to vibrate and produce an audible signal when air passes through, and which air will pass through only when a specific air pressure is reached within the rectum.

13. An air introduction device for use in anastomotic leak testing, comprising a unitary body defining an interior space and comprising
insertion and sealing means for enabling insertion of a part of said body into an anus of a person such that the anus constricts around said part and thereby seals said body against the anal wall,
insertion-limiting means for limiting insertion of said part of said body into the anus and occluding an opening of the anus, and
coupling means for enabling coupling of said body to an insufflation bulb such that air is directable from the insufflation bulb through said coupling means into said interior space in said body.

14. The air introduction device of claim 13, wherein said insertion and sealing means enable insertion of a proximal portion of said body into the anus.

15. The air introduction device of claim 14, wherein said body is substantially tubular and said insertion-limiting means comprise an expanded portion of said body arranged behind said proximal portion and having a larger diameter than said proximal portion, said expanded portion being adapted to engage with the anal opening to limit insertion of said proximal portion into the anus and seal said body against the anal opening.

16. The air introduction device of claim 14, wherein said proximal portion includes a rounded tip to facilitate insertion of said proximal portion into the anus.

17. The air introduction device of claim 13, wherein said coupling means comprise a first lumen arranged on a distal portion of said body and adapted to receive a connector of the insufflation bulb.

18. The air introduction device of claim 17, wherein said distal portion has first and second arms, said first arm defining said first lumen, said second arm defining a second lumen and having a closed distal end, said second lumen being adapted to receive a visualization device when said distal end is opened.

19. The air introduction device of claim 17, wherein said distal portion has first and second arms, said first arm defining said first lumen, said second arm defining a second lumen, further comprising a pressure relief valve arranged in said second lumen for releasing air when a specific air pressure in the rectum is reached.

20. The air introduction device of claim 19, further comprising signal means coupled to said valve for providing a signal when air is released via said valve.

21. The air introduction device of claim 20, wherein said signal means comprise a bill-shaped extension attached to said second arm and having at least one flap arranged to vibrate and produce an audible signal when air is released via said valve.

22. The air introduction device of claim 17 wherein said distal portion has first and second arms, said first arm defining first lumen, said second arm defining a second lumen, further comprising a bill shaped extension attached to said second arm and having at least one flap arranged to vibrate and produce an audible signal when air passes through, and which air will pass through only when a specific air pressure is reached within the rectum.

23. A method for anastomotic leak testing, comprising the steps of:
providing a unitary body defining an interior space and having a proximal portion, a distal portion and an expanded portion interposed between said proximal portion and said distal portion;
coupling an insufflation bulb to the distal portion of the body;
inserting the proximal portion of the body into the anus of a patient to cause the proximal portion to dilate the anus whereby the anus constricts around the proximal portion and a seal is formed between the proximal portion and the wall of the anus and the expanded portion presses against the opening of the anus to prevent the escape of air from the rectum;
filling the pelvis of the patient with fluid;
occluding the bowel proximal to the anastomosis;
compressing the insufflation bulb to distend the rectum of the patient with air; and
checking for anastomotic leaks based on the presence of air bubbles in the pelvic fluid once the rectum is sufficiently distended with air.

24. The method of claim 23, further comprising the steps of:
detaching the insufflation bulb from the body after checking for anastomotic leaks while the proximal portion of the body remains inserted in the anus thereby allowing air from the rectum to escape from the rectum and the rectum to deflate; and
then removing the body from the anus.

25. The method of claim 23, wherein the distal portion has first and second arms, the first arm defining a first lumen and the second arm defining a second lumen and having a closed distal end, the insufflation bulb being received in the first lumen.

26. The method of claim 25, further comprising the steps of:
removing the body from the anus after checking for anastomotic leaks based on the presence of air bubbles in the pelvic fluid once;
severing the closed end of the second arm;
inserting an endoscope into the second lumen so that a tip of the endoscope sits within a space of the body;
coupling the endoscope to a light source and a camera coupled to a video display;
inserting the proximal portion of the body into the anus;
compressing the insufflation bulb to distend the rectum of the patient with air; and
visually inspecting the anastomosis by advancing the tip of the endoscope from the space in the body into the rectum until the anastomosis is viewable through the endoscope to thereby enable the integrity of the anastomosis to be ascertained.

27. The method of claim 26, further comprising the step of rotating the endoscope to view the entire anastomosis.

28. The method of claim 26, further comprising the step of removing the endoscope from the body when the visual inspection is complete while the body is engaged with the anus and then deflating the rectum removing the body out of engagement with the anus.

29. The method of claim 23, wherein the distal portion has first and second arms, the first arm defining a first lumen and the second arm defining a second lumen, the insufflation bulb being received in the first lumen, further comprising the steps of:
arranging a pressure relief valve in the second lumen to allow air to escape from the rectum when a specific air pressure within the rectum is reached.

30. The method of claim 29, further comprising the step of providing a signal when air is released via the valve.

31. The method of claim 30, wherein the step of providing a signal when air is released via the valve comprises the step of attaching bill-shaped extension having at least one flap to the second arm, the at least one flap being arranged to vibrate and produce an audible signal when air is released via the valve.

32. The method according to claim 23 wherein the distal portion has first and second arms, the first arm defining a first lumen and the second arm defining a second lumen, the insufflation bulb being received in the first lumen, further comprising the steps of:
attaching a bill shaped extension having two flaps to the second arm, the flaps being arranged to vibrate and produce an audible signal when air passes between them, and whereby air cannot pass between said flaps until the air pressure in the rectum reaches a specific pressure.

33. An anastomotic leak tester, comprising:
an insufflation bulb having a compressible central portion, a pair of valves on opposite sides of said central portion and arranged to provide a unidirectional flow of air through said central portion upon intermittent compressing of said central portion, and a connector; and
an air introduction device comprising a unitary, elastomeric body defining an interior space and having a proximal portion adapted to be inserted into an anus of a person such that said proximal portion causes the anus to constrict around said proximal portion and thereby seal said proximal portion against the anal wall, a distal portion arranged to mate with said connector of said insufflation bulb to enable air to be directed from said insufflation bulb into and through said body and an expanded portion having a larger size than said proximal portion and interposed between said proximal portion and said distal portion, said expanded portion being adapted to engage with an anal opening to limit insertion of said proximal portion into the anus and seal said body against the anal opening.
